(19) United States
(12) Patent Application Publication
(10) Pub. No.: US 2015/0198610 A9
(43) Pub. Date: Jul. 16, 2015

(54) Lectin Assay for Assessing Glycoforms as an Early Marker in Disease

(71) Applicants: Patrick Robert ROMANO, Doylestown, PA (US); Anand MEHTA, Lansdale, PA (US); Timothy M. Block, Doylestown, PA (US)

(72) Inventors: Patrick Robert ROMANO, Doylestown, PA (US); Anand MEHTA, Lansdale, PA (US); Timothy M. Block, Doylestown, PA (US)

(73) Assignees: Baruch S. Blumberg Institute, Doylestown, PA (US); Drexel University, Philadelphia, PA (US)

(21) Appl. No.: 13/388,971

(22) Filed: Feb. 3, 2012

Related U.S. Application Data
(63) Continuation of application No. PCT/US10/44307, filed on Aug. 3, 2010.
(60) Provisional application No. 61/231,400, filed on Aug. 5, 2009.

(51) Int. Cl. G01N 33/68; C07K 14/42; G01N 33/566

(52) U.S. Cl. CPC G01N 33/6842; C07K 14/42; G01N 2333/42; G01N 2400/00

(57) ABSTRACT

The present invention provides methods, compositions, apparatus, and kits for assessing glycoforms of proteins as a biomarker for disease. A purified recombinant form of lectin is used as a detector molecule to specifically label target sugars expressed in disease states. The high affinity of recombinant lectin allows for automation of assays that would be used in the diagnosis of diseases such as, but not limited to, cancer or liver disease.

### CROSS-REFERENCE

The present application claims the benefit of priority of U.S. Provisional Application No. 61/231,400, filed Aug. 5, 2009, now expired and International Application PCT/US2010/044307, filed Aug. 3, 2010 both incorporated herein by reference.

### BACKGROUND OF THE INVENTION

Various proteins exist in two or more different isoforms that differ only in their pattern of glycosylation. Such differences, or the relative proportions of the differently glycosylated isoforms, may be indicative of a disease or disorder and thus there is a need for assay systems capable of distinguishing between the differently glycosylated isoforms.

The use of antibodies to distinguish between differently glycosylated isoforms of endogenous proteins is problematic as the success rate in raising antibodies which bind specifically or preferentially to particular isoforms of endogenous glycosylated proteins is relatively low.

Further, determination of the relative concentrations of differentially glycosylated isoforms of an endogenous protein has been shown to be clinically important. Keir et al discusses the abnormal relative abundances of the transferring glycosylated isoforms in patients with carbohydrate deficient glycoprotein syndromes or congenital disorder of glycosylation (Keir et al. Ann. Clin. Biochem. 36: 20-36 (1999). Any protein with post-translational glycosylation can potentially occur in different glycosylation isoforms. Thus clinically relevant proteins may exist in different glycosylated isoforms, including glycosylated markers for cancers and other disease, including alkaline phosphatase, alpha-fetoprotein, human chorionic gonadotropoin, and possibly prion protein (CD230).

Other glycoproteins associated with disorders and considered potential targets for assay development in the present invention include, but not limited to, alpha-1-acid glycoprotein, alpha-1-antitrypsin, haptoglobin, thyroglobulin, prostate specific antigen, HEMPAS erythrocyte band 3 (associated with congenital dyserythropoietic anemia type II), PC-1 plasma-cell membrane glycoprotein, CD41 glycoprotein IIb, CD42b glycocalicin, CD43 leukocyte sialoglycoprotein, CD63 lysosomal-membrane-associated glycoprotein 3, CD66a biliary glycoprotein, CD66f pregnancy specific b1 glycoprotein, CD164 multi-glycosylated core protein 24, and the Cd235 glycophorin family.

Aleuria aurantia lectin (AAL) is a 312 amino acid protein having no carbohydrate chain and containing five binding sites for L-fucose or L-fucose-linked oligosaccharides. The multivalent nature of AAL gives it an unusually high binding affinity (micromolar) for carbohydrate ligands compared to other lectins. Commercial production of AAL is based on the isolation and purification of lectin by binding to a fucose-starch column. AAL is eluted from the column with 50 mM L-fucose (Fujihashi et al.). However, structural and biochemical analysis has shown that commercial AAL has 3 to 5 of its 5 ligand binding sites occupied with fucose as a result of this manufacturing process (Olausson et al., Amano et al., Fujihashi et al., and Wimmerova et al.). Consequently, AAL produced through these methods lack the specificity and affinity needed for incorporation into a clinically significant diagnostic assay.

Accordingly, there is a need to have an assay that can selectively distinguish two or more glycoprotein isoforms having different glycosylation patterns, said method comprising contacting a sample containing a glycoprotein expressing two or more glycoforms using a recombinant form of AAL having a high affinity recognition for specific fucosylated oligosaccharides, and directly or indirectly detecting differences in glycoforms as an indicator of disease status.

### BRIEF DESCRIPTION OF THE INVENTION

The present invention incorporates the use of high affinity lectin to detect alterations in target sugar moieties on proteins and provides a means for an automated diagnostic assay in the early detection of diseases. One embodiment of the present invention is a blood test for individuals with inflammatory disorders, autoimmune disorders, cancer, infections, or other disorders where a change in the glycosylation patterns of specific proteins are used as biomarkers in serum or as biomarkers expressed on the surface of cells. Analysis of the levels of these proteins, either through identification of the glycoform or quantification of the protein levels expressing these glycoforms, provides for a simple blood test for detecting people with disease or people at risk for disease progression. An assay incorporating the use of high affinity lectin as a detector in an assay platform such as, but not limited to, a plate-based or bead-based formats is readily automated for clinical or point-of-care environments.

### BRIEF DESCRIPTION OF THE FIGURES

FIG. 1: Overall structure of the AAL monomer with blades 1 through 6. Ellipsoids indicate fucose binding sites 1 through 5 and the corresponding site 6. Three stick modes at sites 1, 2, and 4 show fucose molecules.

FIG. 2: Affinity comparisons for IgG0 Ligand Using Nickel NTA Plates.

FIG. 3: Indirect ELISA using recombinant AAL proteins in competition with commercial AAL proteins.

### DETAILED DESCRIPTION OF THE INVENTION

The present invention is based on the recent discovery that (1) increased serum levels of fucosylated glycoforms of certain proteins serve as early biomarkers of diseases such as cancer and (2) increased serum levels for agalactosylated glycoform of immunoglobulin G, called alpha-gal IgG0, correlates with diagnosis of liver disease (see Example 1). Accordingly, recombinant or mutant forms of lectin (AAL), linked to any reporter molecule known in the art (e.g. a radiolabel, chromophore or fluorophore), are used as an alpha-gal IgG0 detection molecule, specific for fucosylated proteins in blood. Incorporation into an ELISA or bead based assay systems provides the basis for an automated platform to determine a patients disease status.

One embodiment of the present invention considers the production and isolation of a recombinant wild-type AAL protein in E. coli bacteria and/or yeast. Methods are provided that include the creation and production of mutant AAL proteins altered either by site directed mutagenesis or by random mutagenesis and subsequently selected for mutant AAL proteins. These AAL proteins have a high affinity for the outerarm L-fucopyranosyl linkages, more specifically mutated AAL protein having high affinity for the alpha 1-2 outerarm L-fucopyranosyl linkage, alpha 1-3 outerarm L-fucopyranosyl linkage, or alpha 1-4 outerarm L-fucopyranosyl linkage found in serum protein biomarkers in patient with diseases such as, but not limited to, cancer.

A further embodiment of the present invention incorporates the use of core fucose (alpha 1-6 linked) glycoforms as a target for the lectin, either alone or in combination with the outer arm linkages. The core linked fucose is the major disease indicator on a number of serum biomarkers for cancer and specific detection of alpha 1-6 as opposed to outerarm linkages provides a significant biomarker. The N224Q mutant is more selective for the 1-6 linkage than outerarm. This offers a significant advantage over the prior art. Consequently, AAL proteins of the present invention have a high affinity to core alpha 1-6 L-fucopyranosyl linkage found in serum protein biomarkers in patient with diseases such as, but not limited to, cancer.

AAL is a 312 amino acid protein which contains five binding sites for L-fucose or L-fucose-linked oligosaccharides. The multivalent nature of AAL gives it an unusually high binding affinity (micromolar) for carbohydrate ligands compared to other lectins. Commercial production of AAL isolates and purifies the lectin by binding to a fucose-starch column. AAL is eluted from the column with 50 mM L-fucose (Fujihashi et al.). Structural and biochemical analysis has shown that commercial AAL has 3 to 5 of its 5 ligand binding sites occupied with fucose as a result of this manufacturing process (Olausson et al., Amano et al., Fujihashi et al., and Wimmerova et al.).

Surprisingly, it was found that recombinant (r)AAL produced in and isolated from bacteria using nickel affinity chromatography had substantially higher binding affinities for fucosylated oligosaccharides than commercially prepared AAL as determined by surface plasmon resonance studies, tryptophan fluorescence studies and enzyme linked lectin assays.

One embodiment of the present invention is to provide methods for the creation and production of high affinity (recombinant) rAAL and/or mutated rAAL that makes all ligand binding sites available for binding to specific L-fucopyranosyl linkages found in alpha-gal IgG0 and in serum protein biomarkers markers of disease and cancer.

Another embodiment of the present invention is to incorporate the high affinity rAAL as a detector molecule in sensitive and specific blood based assays to determine those at risk for disease such as, but not limited to, cancer.

### Producing and Selecting an High Affinity Mutagenic AAL

Random mutagenesis of AAL is obtained by transforming the AAL-pUC57 plasmid into the XL1 Red mutator E. coli strain (Strategene) following the manufacturers protocol.

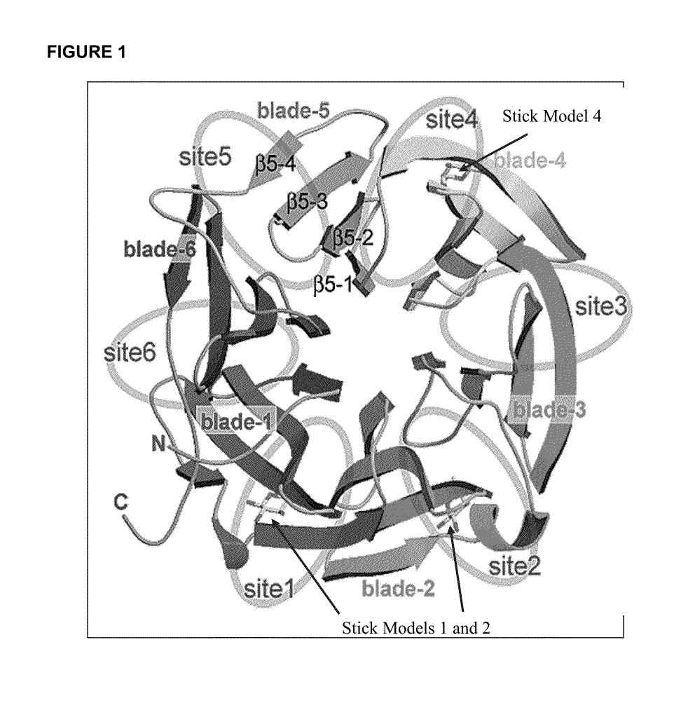

Site directed mutagenesis is focused upon changes in the ligand binding affinities of several of the 5 fucose binding sites in AAL. A beta-propeller motif is iterated six times in AAL. This motif is widely conserved in lectins and constitutes the ligand binding domain. The five (5) sites for fucose binding within the motif (see FIG. 1) have been described with each site having a different affinity for fucosylated ligands.

As shown in FIG. 1, sites 2 and 4 are high affinity ligand binding sites, while sites 1, 3, and 5 have weaker binding affinities. Each beta fold contains highly conserved residues specifically involved in fucose binding. In particular sites 2 and 4 have a conserved glutamine residue at positions Q22 and Q75, respectively, which keeps the fucose binding site in an open fucose accessible state. The corresponding residues in sites 3 and 5 are asparagines N129 and N224. Because these asparagine residues (N) are one carbon shorter than glutamine (Q) they do not make the necessary hydrogen bond contacts with adjacent conserved residues to keep these sites in an energetically stable open configuration. A mutant AAL molecule was developed that change the N129 and N224 residues to glutamines (N129Q, N224Q) individually, AALN129Q and AALN224Q and in combination AALN129Q,N224Q to make these sites structurally resemble the high affinity binding sites.

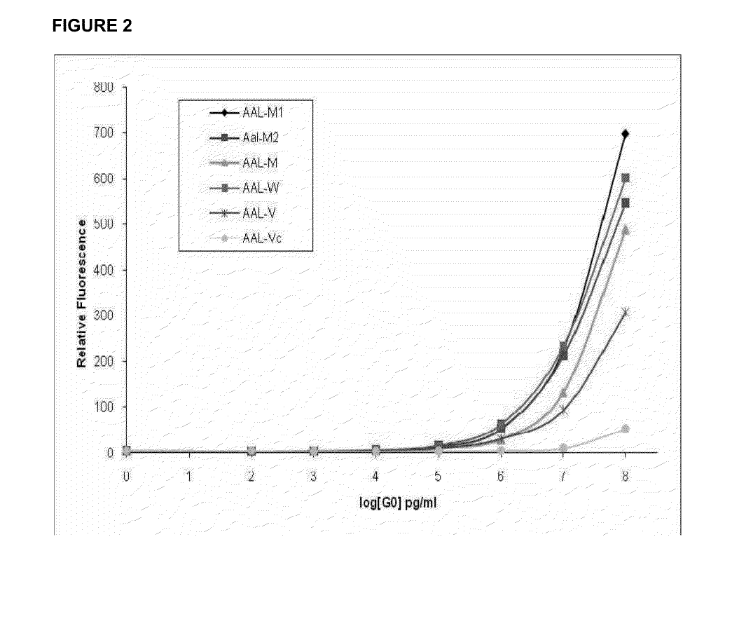

Competition experiments show that these proteins have higher affinity for IgG0 ligands as compared to the wild-type protein (see FIG. 2). These results indicate that the random mutagenesis approach will identify specific high affinity ligand binders to the L-fucopyranosyl linkages used for mutant selection.

Another embodiment of the present invention considers the development of a mutant library of lectins for selection of other sugar structures beyond those specifically mentioned herein.

A yeast (Saccharomyces cerevisiae) based display and selection system is used to screen and select for mutant AAL proteins reactive to specific L-fucopyranosyl linkages. Briefly, a pool of 200-400 XL1 red colonies transformed with AAL-pUC57 and put through the mutator protocol (Strategene) is picked and plasmid DNA prepared. Using standard molecular biology techniques the mutated AAL sequences are subcloned into a version of the yeast display vector pCT302. This system and vector are described in detail in Broder and Wittrup and (2000) Methods Enzymology. Vol. 328, 430-444. Yeast display plasmids containing the mutant AAL library are transformed into a haploid yeast strain EBY100 and grown as described. Mutant AAL proteins are inducibly expressed and displayed on the cell surface of the yeast. The yeast display mutant AAL library is enriched for binding to biotinylated ligand comprising specific L-fucopyranosyl linkages under selection conditions for high affinity binding (Bergan, L., J. A. Gross, B. Nevin, N. Urban, N. Scholler. Cancer Lett 255, 263-74 (2007)). Cells bound to ligand are put through 2 rounds of streptavidin magnetic bead enrichment (Miltenyi Macs) followed by three rounds of flow sorting (Scholler et al., Feldhaus et al., Broder and Wittrup). This system has been shown to obviate expression biases and growth selections common in phage display libraries, and consistently produces rapid enrichment, 10⁵, of selected clones (Feldhaus et al.).

The display library is converted into a secreted mutant AAL library as described by Scholler et al. Mutant AAL DNA is isolated from the enriched display library and cloned by gap repair into the pTOR vector to allow secretion of the mutant AAL proteins into the culture media. Features of the pTOR vector include; an alpha prepro leader to direct secretion of the recombinant protein and in frame additions of a 6× His-tag and an Avitag sequence at the C-terminal end of the mutant proteins (Scholler et al). The Avitag is a biotin acceptor site and is biotinylated at a specific lysine residue when haploid cells transformed with the mutant AAL pTOR library are mated to opposite mating type haploid cells expressing the gene for E. coli biotin ligase (BirA). Diploid cells are selected for on solid media and grown in liquid culture under inducing conditions for secretion of the His-tagged, biotinylated mutant AAL proteins. Diploid cell culture supernatants are desalted, concentrated and buffer adjusted for Nickel-agarose column purification of AAL-His as per manufacturers protocols (His-Trap, Amersham). Recombinant His-tagged, biotinylated mutant AAL can then be purified and used in subsequent ligand binding studies.

Quantitative equilibrium binding of mutant and wild-type AAL for the selection ligand and other L-fucopyranosyl oligosaccharides are determined by BIAcore analysis and enzyme linked lectin assays (Olausson et al).

### High Affinity Recognition of IgG0 Ligand by Mutant AAL Proteins

Equal amounts of recombinant AAL (wild type or mutants) containing 10× Histidine tags were coated to nickel NTA plates and incubated with increasing amounts of IgG0 ligand (X axis). Untagged, commercially purchased AAL (AAL-V) was also coated to nickel NTA plates or on high affinity binding ELISA plates (AAL-Vc) and incubated with increasing amounts of IgG0 ligand. Plates were washed and incubated with fluorescently tagged anti-human IgG, washed and read in a fluorescence detector. Increasing relative fluorescence indicates higher binding affinity. The results showed that the mutant AAL_{M1(N129Q)} exhibited the highest signal to noise ratio, improved the Limit of Detection by 10-100 fold to 10 ng/mL, and increased the dynamic range from 10 ng/mL to 100 ug/mL without saturation (see FIG. 2). These results indicate that recombinant mutant AAL proteins will increase the sensitivity and specificity of a blood based assay to detect subtle changes in serum IgG0 levels.

### Example 1

### rAAL Protein Selective for Cirrhotic Patients

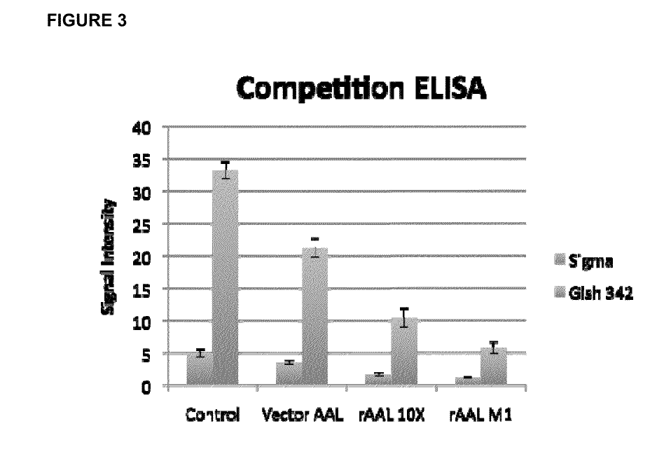

Recombinant lectin (rAAL) was shown to have a higher affinity for the IgG0 ligand than commercially available AAL. In competition ELISA based experiments using serum samples, rAAL had a significantly higher affinity in cirrhotic patients compared with controls (see FIG. 3).

### Experimental Protocol:

96 well plates are coated with sodium periodate (NalO₄) treated mouse IgG (1.0 ug/well). 3 uL of serum from a patient with cirrhosis (Gish 342) or serum from normal donors (Sigma) were diluted to 100 uL and added to antibody coated wells. Plates are washed and then incubated at room temperature for 30 minutes/shaking with equivalent amounts of either unbiotinylated commercial (Vector) AAL, unbiotinylated recombinant 10×-His tagged (rAAL 10×), or unbiotinylated recombinant 10×-His tagged mutant AAL (rAAL N129Q,N224Q)). Equivalent amounts of biotinylated AAL was then added to each well and incubated for 1 hour at room temperature/shaking. Plates are washed and incubated with streptavidin-800-IRDye as previously described. Plates are washed and fluorescence quantified using a LiCor Odyssey machine. Control samples measured biotinylated AAL binding to IgG0 in the absence of unbiotinylated AAL.

### Experimental Results

Competition experiments provide an indirect ELISA for measuring the ability of unbiotinylated commercial (Vector) and unbiotinylated rAAL proteins to interfere with binding of biotinylated AAL to the IgG0 ligand. It is a qualitative indication of the relative affinities of the untagged AAL proteins for the IgG0 ligand.

As shown in FIG. 3, rAAL proteins decrease signal intensity to a larger degree than commercially purchased (Vector) AAL in a competition ELISA based format. In serum samples from a cirrhotic patient (Gish 342) unbiotinylated commercial (Vector) AAL will compete with biotinylated AAL and lead to a decrease in signal intensity compared to control (signal output drops from ˜35 to ˜20). Unbiotinylated wild-type rAAL 10× and a mutant rAAL (N129Q,N224Q) protein lead to greater decreases in signal output versus control, indicating a higher affinity for IgG0 in serum from the cirrhotic patient. Sigma=negative control sera (blood sera from individuals with no liver disease).

A further embodiment of the present invention provides for a kit for an assay method according to the invention, said kit comprising a recombinant or mutant form of AAL having a high affinity recognition for specific fucosylated oligosaccharides.

Although the present invention has been described with reference to specific embodiments, workers skilled in the art will recognize that many variations may be made therefrom, for example in the particular selection of a detection molecule linked to AAL herein described, and it is to be understood and appreciated that the disclosures in accordance with the invention show only some preferred embodiments and advantages of the invention without departing from the broader scope and spirit of the invention. It is to be understood and appreciated that these discoveries in accordance with this invention are only those which are illustrated of the many additional potential applications that may be envisioned by one of ordinary skill in the art, and thus are not in any way intended to be limiting of the invention. Accordingly, other objects and advantages of the invention will be apparent to those skilled in the art from the detailed description together with the claims.

1-10. (canceled)

11. A method for distinguishing two or more glycoprotein isoforms comprising;
a. obtaining a sample containing glycoprotein;
b. binding said glycoproteins to a recombinant lectin containing at least one binding site for L-fucose or L-fucose-linked oligosaccharides and having a mutation to keep the binding site in an energetically stable configuration wherein said lectin has a high affinity for the core linked fucose glycoprotein isoform and wherein said lectin is linked to a reporter molecule; and
c. detecting for the presence of said reporter molecule, the presence of said reporter molecule indicates the presence of the isoform.

12. The method of claim 11 wherein said recombinant lectin is recombinant AAL linked in frame to ten or more histidine residues.

13. The method of claim 11 wherein said recombinant lectin is recombinant AAL having ten or more histidine residues linked in frame to the c-terminus and having an asparagine or glutamine at either position 129 or 224 for detecting said isoform.

14. The method of claim 13 wherein said isoform has an alpha-1,6 linked fucose residue.

15. (canceled)

16. (canceled)

17. (canceled)

18. (canceled)

19. The method of claim 12 further having mutations in at least one of the sites from a group consisting of N129Q in site 3, N224Q in site 5, and combinations thereof where mutated sites 3 and 5 structurally resemble high affinity binding sites.
